(19) United States
(12) Patent Application Publication
(10) Pub. No.: US 2020/0306438 A1
(43) Pub. Date: Oct. 1, 2020

(54) PERITONEAL DIALYSIS MACHINE

(71) Applicant: Fresenius Medical Care Deutschland GmbH, Bad Homburg (DE)

(72) Inventors: Klaus WOLF, Muedesheim (DE); Peter WABEL, Darmstadt (DE)

(73) Assignee: Fresenius Medical Care Deutschland GmbH, Bad Homburg (DE)

(21) Appl. No.: 16/613,443

(22) PCT Filed: May 16, 2018
(86) PCT No.: PCT/EP2018/062802

§ 371 Date: Nov. 14, 2019

(30) Foreign Application Priority Data
May 16, 2017 (DE) 10 2017 110 577.8

(51) Int. Cl. A61M 1/28

(52) U.S. Cl. CPC A61M 1/282; A61M 2205/3334; A61M 2205/3331

(57) ABSTRACT

The invention relates to a peritoneal dialysis machine for the carrying out of a peritoneal dialysis treatment having recurring cycles, the cycles comprising an inflow phase, a dwell period, and a drainage phase for the dialysis fluid, wherein the machine has a control unit and a measurement apparatus for determining the inflow behavior and/or drainage behavior of the dialysis fluid to and from a patient, wherein the control unit is configured to determine a time delay factor on the basis of the measured values collected over a plurality of inflow phases and/or drainage phases, said time delay factor putting a theoretical inflow duration and/or drainage duration into relation with the actual inflow duration and/or drainage duration of dialysis fluid to or from the patient.

The invention relates to a machine for the carrying out of a peritoneal dialysis treatment, in particular an automated peritoneal dialysis treatment, at a patient.

Peritoneal dialysis is also abbreviated to PD. There are various PD processes, including the processes of automated peritoneal dialysis (APD) carried out using peritoneal dialysis machines. In ADP all the treatment steps or at least some of the treatment steps are carried out in an automated manner. Said steps can, for example, be the switching on or off of pumps, the opening or closing of valves, etc.

The effecting of a flow of dialysis solution can take place gravimetrically, i.e. due to gravity, and/or by means of one or more pumps.

The present invention is not restricted to a specific kind of PD, i.e. it comprises automatic machines, non-automatic machines, gravimetric machines and also machines working with pumps.

In PD, the machine supplies a dialysis solution into the abdominal cavity of the patient via a catheter in the inflow phase. This step, like the draining from the abdominal cavity, can take place gravimetrically. The dialysis solution is then left in the abdominal cavity during a dwell period. In this respect, low-molecular substances can pass from the blood via the capillary vessels of the peritoneum into the dialysis solution since a concentration gradient is present. Water can furthermore be removed from the body in this manner provided that the dialysis solution has a higher content of osmotically active substances than the blood. After the end of the dwell period, the machine removes the dialysis solution enriched with eliminated substances and consequently used up in a drainage phase from the abdominal cavity again via the catheter. The fluid exchange can take place gravimetrically or actively using a pump.

The cycle of inflow phase, dwell period and drainage phase is repeated several times in typical process management routines, for example over night while the patient is asleep. A new inflow phase is always started whenever the machine determines that the drainage phase has ended, i.e. that the used dialysis fluid has been completely drained from the abdominal cavity of the patient.

It is known in the prior art to provide an estimate of the total treatment duration of a peritoneal dialysis treatment comprising a plurality of cycles in the interest of better planning security for carers and patients. It is, for example, disclosed in DE 10 2014 005 122 A1 to estimate the total treatment duration in that the ideal treatment duration is taken as a starting point and is incremented in a specific manner. Time delay factors for the inflow duration and the drainage duration of dialysis fluid to or from the patient are inter alia provided that are intended to reflect a non-ideal behavior of the catheter (for example a factor or multiplier of 1.5). Such time delay factors were, however, previously only estimated or were set in a manner not specific to a patient, whereby the estimation of the total treatment duration could indeed be improved, but the result still suffered from substantial uncertainties. A possible continuous deterioration of the catheter quality due to clogging with fibrin or due to a change of position of the catheter within the patient (within the abdominal cavity or Douglas cavity) has previously not been taken into account.

It is the object of the invention to provide a peritoneal dialysis machine with which a more reliable estimation of the total treatment duration can be carried out.

Against this background, the invention relates to a peritoneal dialysis machine for the carrying out of an automated peritoneal dialysis treatment having recurring cycles, the cycles comprising an inflow phase, a dwell period and a drainage phase for the dialysis fluid, wherein the machine has a control unit and a measurement apparatus for determining the inflow and/or drainage behavior of the dialysis fluid to and from a patient. Provision is made in accordance with the invention that the control unit is configured to determine a time delay factor on the basis of the measured values collected over a plurality of inflow phases and drainage phases, said time delay factor putting a theoretical inflow and/or drainage duration into relation with the actual inflow and/or drainage duration of dialysis fluid to and from the patient.

The invention is therefore directed to the determination of a time delay factor individual to the patient. This time delay factor can be determined, for example, using a plurality of measured values or value progressions (curves) that were measured in a specific number of preceding cycles or treatments. It can therefore be a mean value individual to the patient.

Provision is made in an embodiment that the machine is a gravimetrically working machine. One or more valves can be provided, for example, and the control unit can be configured such that the or a valve is opened after the dwell phase and before the start of the drainage phase to enable a gravimetric drainage of dialysis fluid from the patient and/or such that the or a valve is opened after the drainage phase and before the start of the inflow phase to enable a gravimetric inflow of dialysis fluid to the patient.

Provision is made in an embodiment that the machine has a dialysis pump that is configured to pump dialysis fluid from and/or to the patient. It can therefore be an actively conveying machine in which the control unit is configured such that dialysis fluid is removed from the patient during the drainage phase using the dialysis pump and/or such that the patient is supplied with dialysis fluid during the inflow phase using the pump.

Provision is made in an embodiment that the measurement apparatus is configured to determine the flow rate of the dialysis fluid. A flow sensor can therefore be provided by means of which the time progression of the flow rate during the inflow phase and/or drainage phase can be determined and recorded in the control unit. Provision can be made in this embodiment that the control unit is configured to determine the time delay factor on the basis of the measured values for the flow rate collected over a plurality of inflow phases or drainage phases. The time progression of the flow rate or the flow rate itself can also take place gravimetrically by the weighing of the solution or of a bag or of another vessel by means of a scale at two different points in time. A conclusion on the flow rate can also be drawn from this or the flow rate can be calculated from this.

Provision is made in an embodiment that the measurement apparatus is configured to determine a hydrostatic pressure in the line or a pressure difference at a dialysis pump. A pressure sensor can therefore be provided by means of which the progression of the hydrostatic pressure in the line, in particular with gravimetric systems, or the pressure drop at a dialysis pump, in active systems or in systems having at least one pump, can be determined during the inflow and/or drainage phase and can be recorded in the control unit. Provision can be made in this embodiment that the control unit is configured to determine the time delay factor on the basis of the measured values for the pressures or for the pressure differences collected over a plurality of inflow phases or drainage phases. The pressure differences can be recorded in the control unit as functions of time or also as functions of the filling volume. To this extent, the machine can have a measurement apparatus for both the flow rate and the pressure or the pressure difference and provision can be made that the time delay factor is determined on the basis of both of these measured values.

Provision is made in an embodiment that the control unit is configured to provide an estimate of the total treatment duration for a specific prescription, wherein a theoretical inflow duration and/or drainage duration is multiplied by the time delay factor in the preparation of the estimate. Provision can, for example, be made that the determination of the ideal total treatment duration comprises the multiplication of an ideal cycle duration by the number of cycles. The determination of the real total treatment duration accordingly comprises the multiplication of a real cycle duration by the number of cycles. The ideal cycle duration is determined from the sum of the idealized duration of the inflow phase, from the idealized duration of the dwell phase, and from the idealized duration of the drainage phase. The real cycle duration is determined correspondingly from the sum of the real duration of the inflow phase, of the real duration of the dwell phase, and of the real duration of the drainage phase. The real duration of the inflow phase can now be obtained in accordance with the invention by multiplication of the idealized duration of the inflow phase by the time delay factor. The real duration of the drainage phase can accordingly be obtained in accordance with the invention by multiplication of the idealized duration of the drainage phase by the time delay factor.

Provision is made in an embodiment that the control unit is configured such that a signal is output when the time delay factor exceeds a threshold value. Provision can be made for this purpose that the machine has a signal unit or an interface for communication with an external signal unit. Suitable signals, for example, comprise visual signals, audio signals or vibration signals. A decision can be made, for example, on the basis of a signal output whether it could make sense to change the prescription in view of the time circumstances. A decision can furthermore be made, for example, whether it could make sense to replace the catheter. An evaluation on the basis of graphical or tabular records in suitable software is thus made possible.

Provision is made in an embodiment that the control unit is configured such that a treatment parameter is modified when the time delay factor exceeds a specific threshold value. Provision can therefore be made to better adapt treatment parameters to the existing real conditions using the time delay factor.

Provision is made in an embodiment that the modified treatment parameter is a criterion for fixing the end of a drainage phase, preferably a minimal outflow rate. Provision can be made that the control unit is configured to make the determination whether a drainage phase has ended and a new inflow phase can begin using preset criteria, for example using a reached minimal drainage volume and/or using the falling below of a specific flow rate.

Provision is made in an embodiment that the modified treatment parameter is the pumping rate of a dialysis pump that is configured to pump dialysis fluid from and/or to the patient. Provision can be made in this respect that a starting value for the pumping rate is modified, for example the pumping rate that is set at the start of a drainage phase. Provision can furthermore be made that a progression profile for the pumping rate is modified; for example, the change of the initial pumping rate in the course of a drainage phase.

At least one memory can furthermore be provided in which the time delay factor is stored in the treatment protocol to enable a further processing, preferably a graphical treatment of the time delay factor over a plurality of treatments. The memory can represent a component of the machine or can also be designed as an external memory.

The invention furthermore comprises a method of carrying out a peritoneal dialysis using a peritoneal dialysis machine in accordance with the invention, wherein a time delay factor is determined on the basis of the measured values collected over a plurality of inflow phases and drainage phases, said time delay factor putting a theoretical inflow duration and/or drainage duration into relation with the actual inflow duration and/or drainage duration of dialysis fluid to and from the patient. Advantageous embodiments of the method result from the above description of the configuration of the control unit in the peritoneal dialysis machine in accordance with the invention.

Further details and advantages of the invention result from the embodiment explained in the following with reference to the Figures. There are shown in the Figures:

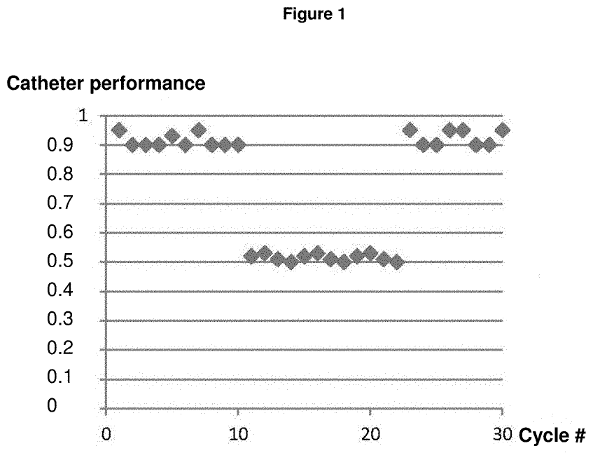

FIG. 1: a possible progression of the catheter quality in the course of a plurality of treatments;

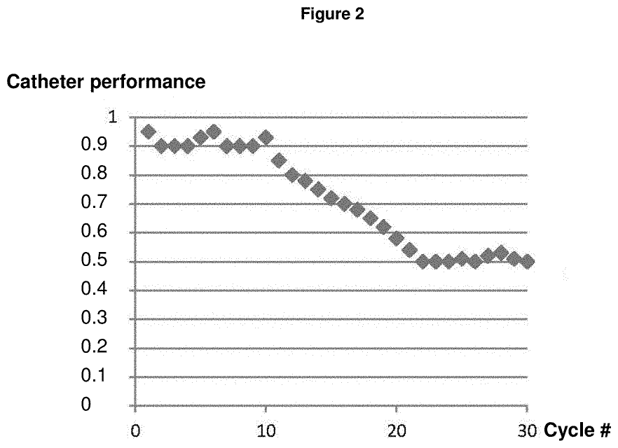

FIG. 2: another possible progression of the catheter quality in the course of a plurality of treatments; and

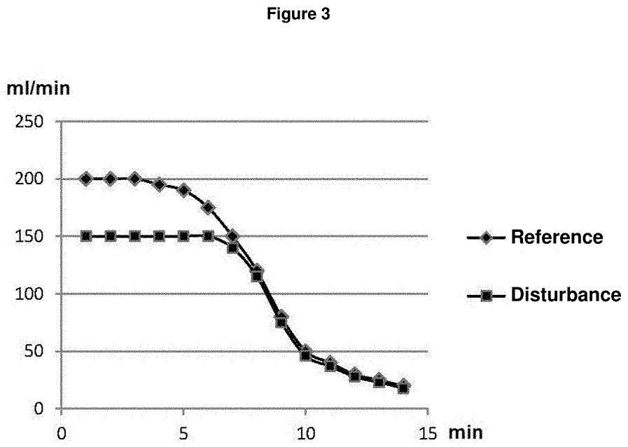

FIG. 3: possible time progressions of the drainage rate (volume/time) of the dialysis fluid out of the abdominal cavity during the drainage phase.

In accordance with an embodiment, a peritoneal dialysis machine is provided that is intended for the carrying out of an automated peritoneal dialysis treatment having recurring cycles comprising an inflow phase, a dwell period, and a drainage phase for the dialysis fluid. The machine comprises a control unit, a measuring apparatus for determining the inflow rate and the drainage rate of the dialysis fluid from a patient, and a dialyzate pump for conveying dialysis fluid to and from the patient.

An algorithm is stored in the control unit with reference to which a determination of the real total duration period (Δt_{total,real}) can be carried out. The algorithm is based on a multiplication of a real cycle duration (Δt_{cycle,real}) by the number of cycles (n), supplemented by a real duration of an initial drainage phase (Δt_{init,real}) and a real duration of a final inflow phase (Δt_{fin,real}).

Δ_{total,real}=Δt_{cycle,real}×n+Δt_{init,real}+Δt_{fin,real}

The real cycle duration (Δt_{cycle,real}) is determined from the sum of the real inflow duration (Δt_{in,real}), of the real dwell duration (Δt_{dwell,real}), and of the real drainage duration (Δt_{drn,real}).

Δ_{cycle,real}=Δt_{in,real}+Δt_{dwell,real}+Δt_{drn,real}

The present invention deals with the exact determination of the real inflow duration (Δt_{in,real}), of the real drainage duration (Δt_{drn,real}), of the real duration of the initial drainage phase (Δt_{init,real}) and of the real duration of the final inflow phase (Δt_{fin,real}). The duration of these real phases is determined in accordance with the invention from the multiplication of the corresponding ideal durations (Δt_{in,ideal}, Δt_{drn,ideal}, Δt_{init,ideal} and Δt_{fin,ideal}) that can be calculated with known machine configurations by a time delay factor F. Different time delay factors are provided for inflow processes (Δtᵢₙ, Δt_{fin}) and for the drainage processes (Δt_{drn}, Δtᵢₙᵢₜ), namely the factors F_{drn} and Fᵢₙ.

Δt_{in,real}=Δt_{in,ideal}×Fᵢₙ

Δt_{drn,real}=Δt_{drn,ideal}×F_{drn}

Δt_{init,real}=Δt_{init,ideal}×F_{drn}

Δt_{fin,real}=Δt_{fin,ideal}×Fᵢₙ

The time delay factors F_{drn} and Fᵢₙ are determined by the control unit using a plurality of measured progressions of the drainage rates and inflow rates that were measured in a specific number of preceding cycles (for example 20 cycles). It is therefore a mean value that is individual to the patient and that is representative of the catheter quality.

Possible progressions of the catheter quality in the course of a plurality of treatments are shown in FIGS. 1 and 2. The abscissa designates the running number of a cycle of inflow phase, dwell phase and drainage phase. The ordinate designates a value representative of the catheter performance that can, for example, be determined using the flow at a specific pressure difference. The value 1 stands for an ideal catheter performance; values under 1 for a correspondingly reduced catheter performance. In FIG. 1, an abrupt, significant drop in the catheter performance can be observed after a specific number of cycles, which can be due, for example, to an abrupt change of the positioning of the catheter in the abdominal cavity of the patient. A gradual drop in the catheter performance is illustrated in FIG. 2 such as can be observed, for example, by a continuous clogging of the catheter with fibrin.

In the machine in accordance with the embodiment, the control unit is furthermore configured to output a signal when a time delay factor F_{drn} or Fᵢₙ exceeds a threshold value stored in the control unit. The machine comprises an interface for communication with an external computer for this purpose. A decision can be made, for example, on the basis of a signal output whether it could make sense to change the prescription in view of the time circumstances. A decision can furthermore be made, for example, whether it could make sense to replace the catheter, to check or to correct the position of the catheter or optionally to flush the catheter. The evaluation takes place on the basis of graphical and tabular records in suitable software.

The control unit is furthermore configured such that the value for a drainage rate used as a criterion for determining the end of a drainage phase is corrected downward by a specific amount at the end of the drainage phase when the time delay factor F_{drn} exceeds the threshold value stored in the control unit. FIG. 3 shows time progressions of the drainage rate in a peritoneal dialysis machine. The curve “Reference” shows a time progression of the drainage rate in the normal case. The drainage rate additionally adopts a value of approximately 200 ml/min and falls from about minute 5 due to the hydrostatic pressure becoming lower. The curve “Disturbance” shows a time progression of the drainage rate with an impaired catheter performance. The drainage rate initially only adopts a value of approximately 150 ml/min. A reduction after approximately minute 5 is, however, also observed here so that after approximately 10 minutes substantially less liquid has drained. If suitable countermeasures are not taken, this would have the consequence of a worse treatment quality and of a longer treatment duration. The correction of the threshold value counteracts this problem.

The starting value and the change progression of the rate of the dialysis pump in the inflow phase or the outflow phase are furthermore also changed when the time delay factor Fare or Fin exceeds the threshold value stored in the control unit. It is thus possible additionally to counteract a falling treatment quality.

Advantages of the solution in accordance with the invention, for example, comprise the possibility of a more reliable estimate of the total treatment duration of a peritoneal dialysis treatment with a given prescription. The invention furthermore enables the quality of the catheter to be observed over the course of a plurality of treatments and also, for example, to graphically visualize it. An early recognition of changes to the catheter is possible. The treatment parameters and/or system parameters can be better adapted to an individual situation or to an individual patient in an embodiment of the invention. The necessity of a correction of the prescription can also be recognized with the aid of the solution in accordance with the invention in an embodiment. Changes in the condition of the patient can be recognized. Messages and responses to the patient relating to different behavior scenarios can be improved.

1. A peritoneal dialysis machine for the carrying out of a peritoneal dialysis treatment having recurring cycles, the cycles comprising an inflow phase, a dwell period and a drainage phase for the dialysis fluid, wherein the machine has a control unit and a measurement apparatus for determining the inflow and/or drainage behavior of the dialysis fluid to and from a patient, characterized in that
the control unit is configured to determine a time delay factor on the basis of the measured values collected over a plurality of inflow phases and/or drainage phases, said time delay factor putting a theoretical inflow and/or drainage duration into relation with the actual inflow and/or drainage duration of dialysis fluid to and from the patient.

2. A peritoneal dialysis machine in accordance with claim 1, characterized in that the machine is a gravimetrically working machine.

3. A peritoneal dialysis machine in accordance with claim 1, characterized in that the machine has a dialysis pump that is configured to pump dialysis fluid from and/or to the patient.

4. A peritoneal dialysis machine in accordance with claim 1, characterized in that the measurement apparatus is configured to determine the flow rate of the dialysis fluid.

5. A peritoneal dialysis machine in accordance with claim 1, characterized in that the measuring apparatus is configured to determine a hydrostatic pressure in the line or a pressure difference at a dialysis pump of a peritoneal dialysis machine not working purely gravimetrically.

6. A peritoneal dialysis machine in accordance with claim 1, characterized in that the control unit is configured to output an estimate of the total treatment duration for a specific prescription, with a theoretical and/or calculated inflow duration and/or drainage duration being multiplied by the time delay factor in the preparation of the estimate.

7. A peritoneal dialysis machine in accordance with claim 1, characterized in that the control unit is configured such that a signal is output when the time delay factor exceeds a threshold value.

8. A peritoneal dialysis machine in accordance with claim 1, characterized in that the control unit is configured such that a treatment parameter and/or a system parameter is/are modified when the time delay factor exceeds a specific threshold value.

9. A peritoneal dialysis machine in accordance with claim 8, characterized in that the modified treatment parameter is a criterion for fixing the end of a drainage phase, preferably a minimal outflow rate.

10. A peritoneal dialysis machine in accordance with claim 8, characterized in that the modified treatment parameter is the pumping rate of a dialysis pump that is configured to pump dialysis fluid from and/or to the patient.

11. A peritoneal dialysis machine in accordance with claim 1, characterized in that at least one memory is provided in which the time delay factor is stored in the treatment protocol to enable a further processing, preferably a graphical processing of the time delay factor over a plurality of treatments.
